(19) United States
(12) Patent Application Publication
(10) Pub. No.: US 2017/0283382 A1
(43) Pub. Date: Oct. 5, 2017

(54) METHOD FOR PRODUCING PURE L-HERCYNINE

(71) Applicant: TETRAHEDRON, Paris (FR)

(72) Inventor: Irène ERDELMEIER, Paris (FR)

(21) Appl. No.: 15/509,054

(22) PCT Filed: Sep. 18, 2015
(86) PCT No.: PCT/FR2015/052502

§ 371 Date: Mar. 6, 2017

(30) Foreign Application Priority Data
Sep. 19, 2014 (FR) 1458869

(51) Int. Cl. C07D 233/64; C07B 59/00

(52) U.S. Cl. CPC C07D 233/64; C07B 59/002; C07B 2200/05

(57) ABSTRACT

The invention relates to a method for the purification of L-hercynine. Said method for the purification of L-hercynine, from a reaction mixture resulting from the reaction of L-histidine, in controlled pH conditions, with a methylating agent Me X in a polar solvent or a mixture of polar solvents, at room temperature, is characterised in that it comprises at least one step of separating the organic products from the inorganic salts formed during the reaction by electrodialysis. This method allows the L-hercynine losses to be limited during the purification.

The present invention relates to a method for the industrial-scale production of pure L-hercynine.

This invention relates to a new method for obtaining pure L-hercynine without any chromatography purification step. More particularly, this invention relates to a new industrial method for producing pure L-hercynine that includes a step for electrodialysis purification, as well as a new method for producing L-hercynine from L-histidine, as well as a new L-hercynine-d9 compound.

### BACKGROUND OF THE INVENTION:

Compounds of the betaine (quaternary ammonium) type are among the most abundant nitrogenous organic molecules in the ground after proteogenic and non-proteogenic amino acids. Glycine betaine and hercynine are the main representatives of these betaines. L-hercynine is the betaine derivative of L-histidine.

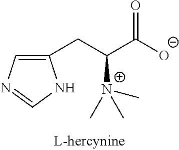

L-hercynine is the direct precursor of L-ergothioneine (Y. Ishikawa et al., J. Biol, Chem.; 1974; 249; 4420-4427) in certain fungi, mycobacteria and cyanobacteria (Pfeiffer, C. et al.; Food Chemistry; 2011; 129; 4; 1766-1769). It has therefore naturally been found in these microorganisms (Mahmood, Z.A. et al.; Pak. J. Pharm. Sci.; 2010; 23; 349-357), but also in some snakes (Ackermann, D. et al.; Z. Physiol. Chem. Hoppe-Seyler's; 1960; 318; 212-217). Very recently, L-hercynine was shown for the first time in human red blood cells (Chaleckis, R. et al.; Mol. Biosystems; 2014; DOI 10.1039/c4mb00 346b).

L-hercynine is a growth regulating substance in fungi such as Agaricus bisporus (Champavier, Y. et al.; Enz. and Microbial. Technologt; 2000; 26; 2-4; 243-251). As a betaine, L-hercynine could play an osmolyte role, in particular in plants (Velisek, J.; Chemistry of Food; J. Wiley & Sons Eds.; 2013). As such, L-hercynine performs a cyto-protective function in particular by contributing to the correct folding of proteins in the cells.

Although its biological characteristics are considerable, L-hercynine has not been studied very much biologically; this is probably due to the fact that it is not commercially available (Warren, C. R.; New Physiologist; 2013; 198; 476-485).

Regarding its preparation, whether direct (1 step) or indirect (2 steps), only 4 documents published to date mention it on the laboratory scale.

In 1968, Melville and his team reported the first synthesis of L-hercynine from L-histidine in 2 steps (Reinhold, V. et al.; J. Med. Chem.; 1968; 11; 258-260).

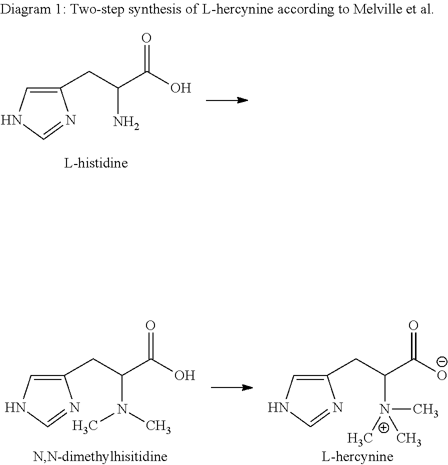

During the first step, the L-histidine undergoes methylation under reducing conditions using formaldehyde in the presence of palladium on carbon and hydrogen to lead to L-N,N′di-methyl-histidine. After treatment using organic solvents, the desired intermediary is obtained by recrystallization with a yield of 82%. The second step consists of methylation using methyl iodide with pH 9. The L-hercynine is obtained after treatment and purification on ion exchange column, then recrystallization with a yield of 89%. It should be noted that the conditions used by Melville are not racemizing. The global synthesis yield of pure L-hercynine from L-histidine using this procedure is therefore 73%. Having tested the direct tri-methylation route for L-histidine, Melville mentions “difficulties in the tests to prepare L-hercynine by direct methylation of L-histidine in particular due to the concomitant methylation of the imidazole core that occurs under the conditions used and the corresponding pentamethylated derivative that forms during the reaction” (Reinhold, V. et al.; J. Med. Chem.; 1968; 11; 260).

More recently, a second document mentions hercynine as a non-isolated intermediary in the production of urocanic acid, the desired objective for the authors of this publication (Valeev, F.A. et al., Chemistry of Natural Compounds; 2007; 43(2); 143-148) according to:

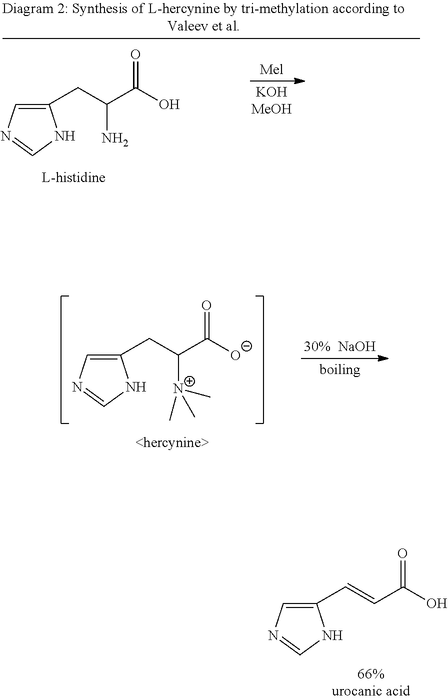

The authors mention the “one-step” methylation of L-histidine to obtain, on an intermediary basis, hercynine, which, in a highly basic medium and hot, leads, after elimination of the trimethyl amine, to the desired urocanic acid with a global yield of 66% from the L-histidine. Not having isolated the intermediate hercynine, or shown the trimethyl amine as a sub-product, the formation of the latter is only hypothetical. Furthermore, at no time do they mention sub-products originating from the methylation of the imidazole core, as mentioned by Melville in his 1968 publication. Lastly, since the chiral center is destroyed during the reaction sequence leading to urocanic acid, no information is available in order to determine whether this direct tri-methylation is done with or without racemization.

In 2012, the preparation (in solution and mixed with the per-methylation products) of hercynine by direct methylation for analytical studies was done using a very large excess (16 equivalents) of methylation agent (Chary V. N. et al., J. Mass. Spectrom. 2012; 47; 79-88).

Lastly, in a Master's thesis in chemistry, Khonde also mentions the preparation of L-hercynine by direct methylation of the L-histidine, but without purifying the desired L-hercynine (Khonde, L.P.; Synthesis of Mycobacterial ergothioneine biosynthetic pathway metabolites; University of Cape Town, South Africa; 2013; page 61, diagram 3.19).

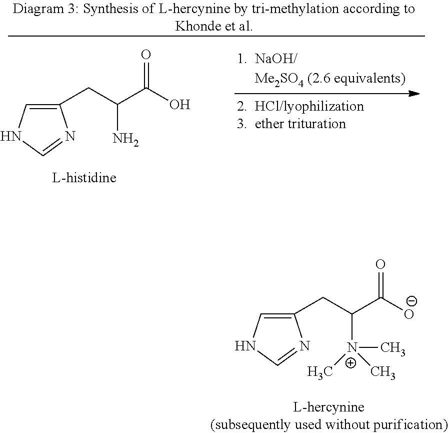

Khonde uses dimethyl sulfate, as methylating agent, in a basic medium (10% NaOH). Curiously, only 2.6 equivalents of dimethyl sulfate are used, whereas at least 3 equivalents are theoretically necessary to obtain L-hercynine from L-histidine. Furthermore, no structural data is provided by the author, who uses the raw reaction medium for the following step.

Although there are several chemical synthesis methods for L-hercynine in solution and in mixture with permethylation products, as we saw above, the obtainment of pure L-hercynine is severely limited by the difficulties encountered during its purification. Indeed, the most used purification technique for amino acids, well known by those skilled in the art, i.e., recrystallization, is not very effective or completely unusable in the presence of large quantities of salts. Only ion exchange chromatography purification methods make it possible to obtain L-hercynine with a good purity. In the context of the two-step preparation described by Melville and already cited in this document (Reinhold, V. et al.; J. Med. Chem.; 1968; 11; 258-260), to obtain a kilogram of pure L-hercynine, it would be necessary to use 13.2 kg of ion exchange resins. On an industrial scale, this represents considerable quantities of resins that must be eliminated in accordance with environmental regulations. This highlights the importance of being able to have a purification method that avoids using very large volumes of resins.

In the Journal of Medicinal Chemistry vol. 11, no. 2, March 1, 1968, pages 258-260, REINHOLD et al. also describe a method for producing Hercynine from alpha-N,N-dimethyl-L-histidine.Hcl.H2O histidine.Hcl.H20 in the methanol adjusted to 9, through the addition of NH4OH, and reaction with Mel at room temperature overnight.

The solvent is next evaporated under reduced pressure to yield a solid white residue that is subsequently treated on an ion exchange column while being re-dissolved in a minimal amount of water.

Furthermore, in Green Chemistry, 2012, 14, pages 2256-2265, ERDELMEIER et al. describe a desalination method for the reaction medium containing the ergothioneine by electrodialysis. ERDELMEIER stresses that electrodialysis has advantages relative to the ion exchange column and leads to a high purity of the ergothioneine.

However, it was not obvious to replace the ion exchange column purification method of REINHOLD with the electrodialysis method used by ERDELMEIER, even though ergothioneine and hercynine have neighboring structures.

Indeed, first of all, the ERDELMEIER document mentions the use of electrodialysis, but stresses that this is done after the cysteine precipitation (“cysteine scavenging”) step (see page 2259, right column, 2^{nd} §).

Yet it is clearly specified previously on the same page 2259, left column, that the cysteine precipitation occurs just after adjusting the pH to 4-5.

In the context of the first tests done by the inventors with electrodialysis, at an initial pH of about 7, losses of 18-25% of the hercynine are observed; see comparative examples 1 and 2 at the end of this description.

However, surprisingly, it was discovered in the context of the present invention that if the pH is at least 8, in particular about 9, the hercynine losses are significantly lower than 5%.

This unexpected drop in hercynine losses was not at all obvious for one skilled in the art. It should also be emphasized that in the ERDELMEIER document, nothing is said about the pH at the beginning of the electrodialysis, and in particular nothing is said about its relevance to the loss level.

It should also be noted that the ERDELMEIER document mentions a global yield of 40% in ergothioneine over the two steps (see page 2259, right column 2^{nd} §).

ERDELMEIER thus demonstrates that although the electrodialysis results in a very high purity, the product yield, i.e., ergothioneine, is obtained with a global yield of 40%, demonstrating major losses of ergothioneine to one skilled in the art, which are unacceptable on an industrial scale.

Under these conditions, it was not obvious for one skilled in the art to find conditions for hercynine with losses <5%, as claimed in the context of the present invention.

Although its physiological role is still poorly defined, L-hercynine is a natural molecule whose significance appears obvious. It is therefore essential to be able to have a method for effectively producing pure L-hercynine that is easy to industrialize, economically viable, does not cause racemization, and respects the environment.

Knowing that recrystallization is not very effective, or is even completely unusable, in the presence of a large quantity of salts, several difficulties must be resolved beyond the problem relative to the stereochemistry of the final product, namely:
1. the complete separation of the L-hercynine, a very hydrophilic molecule, and inorganic salts, sub-products of the methylation reaction (direct or indirect) through an economically viable method that can be industrialized, excluding ion exchange chromatography;
2. an implementation limiting operational losses of the desired product, i.e., <5% loss of L-hercynine during the desalination step, and lastly
3. obtaining pure L-hercynine from a mixture containing structurally very close derivatives such as L-N-mono-methyl-histidine, L-N,N-di-methyl-histidine, or products from the permethylation as mentioned by Melville.

### Aims of the Invention:

One of the aims of the present invention is to propose a simple and reproducible method for producing pure L-hercynine, or its enantiomer, D-hercynine, or its racemic mixture, or one of its isotopically marked derivatives, in particular including a purification step allowing easy separation of the L-hercynine from the formed inorganic salts, before the elimination of the organic sub-products.

These aims are achieved owing to the present invention, which is based on a method for separating L-hercynine from the inorganic salts present in the reaction medium, by electrodialysis, irrespective of the mode used for methylation of the L-histidine or one of its methylated derivatives, followed by purification by recrystallization. This is exemplified in the present invention.

### Brief Description of the Invention:

The present invention therefore aims to resolve the new technical problem consisting of providing pure L-hercynine, or its enantiomer, D-hercynine, or its racemic mixture, or one of its isotopically marked derivatives, according to a solution that encompasses a step for purification of the L-hercynine other than ion exchange chromatography.

The Applicant has developed a new method for producing pure L-hercynine that meets all of these criteria, for the first time simultaneously resolving all of the stated technical problems, very easily and having many advantages over the existing methods. In particular, the invention makes it possible to manufacture large quantities allowing use of the method on an industrial scale. An industrial scale refers to a scale ≧1 kg, in particular greater than 5 kg, still better greater than 10 kg.

Thus, in its first aspect, the present invention relates to a method for purifying L-hercynine, or its enantiomer, D-hercynine, or its racemic mixture, or one of its isotopically marked derivatives from a reaction mixture resulting from the reaction of L-histidine, or its enantiomer D-histidine or its racemic mixture, or one of its isotopically marked derivatives or one of its α-N-methylated derivatives, under controlled pH conditions, with a methylation agent MeX in a polar solvent or a mixture of polar solvents, at room temperature, characterized in that it comprises at least one step for separating the organic products from the inorganic salts formed during the reaction by electrodialysis; and in that the electrodialysis is conducted on a reaction medium adjusted to a pH comprised between 8.5 and 9.5, at the beginning of the electrodialysis.

According to one particular embodiment, the invention relates to a purification method characterized in that it comprises an additional step for recrystallization of the L-hercynine, or its enantiomer, D-hercynine, or its racemic mixture, or one of its isotopically marked derivatives or one of its methylated derivatives, to eliminate the organic impurities.

According to a second aspect, the invention relates to a method for the industrial-scale production of L-hercynine, or its enantiomer, D-hercynine, or its racemic mixture, or one of its isotopically marked derivatives, or one of its α-N-methylated derivatives, characterized in that it comprises the following steps:
reacting the L-histidine, or its enantiomer D-histidine or its racemic mixture, or one of its isotopically marked derivatives or one of its α-N-methylated derivatives, under controlled pH conditions, with a methylation agent MeX in a polar solvent or a mixture of polar solvents, at room temperature; followed by
separating the organic products from the inorganic salts formed during the reaction by electrodialysis and in that the electrodialysis is done on a reaction medium adjusted to a pH comprised between 8.5 and 9.5, upon starting the electrodialysis; followed by
re-crystallizing the L-hercynine to eliminate the organic impurities.

The α-N-methylated derivatives of L-histidine refer to L-N-mono-methyl-histidine or L-N,N′ dimethylhistidine or one of their salts.

Room temperature refers to a temperature comprised between 20° C. and 35° C.

For each aspect of the invention, it is possible to carry out the following particular embodiments:

According to one particular embodiment, the method according to the invention is characterized in that the electrodialysis is done on a reaction medium adjusted to about 9, at the beginning of the electrodialysis. The pH can be adjusted traditionally by adding an inorganic base such as sodium hydroxide, potassium or lithium hydroxide.

It has in fact surprisingly been discovered that hercynine losses during electrodialysis may be reduced to <5% if the pH of the aqueous medium is adjusted to 8.5 to 9.5 at the beginning of the electrodialysis, whereas outside this range, for example at about 7, hercynine losses of 18-25% are observed.

The electrodialysis according to the invention also makes it possible to achieve complete desalination. Complete desalination refers to the separation of the salts from the organic product in aqueous solution until a salt proportion is obtained of less than 1% by weight, which generally corresponds to a final conductivity of <200 μS/cm.

It is equally surprising for one skilled in the art that the electrodialysis method is applicable to the complete separation of the inorganic/mineral salts obtained during the purification or preparation step of the L-hercynine, as betaine, or its enantiomer D-hercynine or its racemic mixture, or of one of its isotopically marked derivatives, and without significant product loss (<5%). Although electrodialysis is known by those skilled in the art as a high-performing and very cost-effective desalination method, significant losses by transmembrane migration have nevertheless been observed for certain organic compounds, such as amino acids (Eliseeva T. V. et al., Desalination; 2002; 149; 405-409, Sato et al., J. Membrane Sci.; 1995; 100; 209-216), and in particular for betaines (EP 2,216,088; WO 9808803).

In general, these losses, caused by diffusion, facilitated electromigration or osmotic pressure, are even greater if complete desalination (<1% salts) is sought, characterized by low final conductivity (<200 μS/cm) and limit current (Shaposhnik V. A. et al., J. Membrane Sci. 1999; 161; 223-228) values. For the industrial desalination of betaines such as 4-butyobetaine by electrodialysis, it has even been proposed to combine the electrodialysis with pretreatment of the effluents in order to recover the lost betaine (EP 2,216,088).

According to another specific embodiment, the method according to the invention is characterized in that the methylating agent MeX is chosen from among methyl halogenide, methyl sulfate, methyl carbonate or methyl or trifluoromethyl or tosyl sulfonate.

According to still another specific embodiment, the method according to the invention is characterized in that the polar solvent or the mixture of polar solvents is chosen from among water; a C1—C6 alcohol, for example methanol, ethanol, propanol or propanol-2, butanol; ethyl acetate. One particular polar mixture is a water/alcohol, water/propanol or propanol-2, methanol/ethyl acetate mixture.

According to another specific embodiment of the method according to the invention, the controlled pH conditions are chosen to result directly in a tri-methylation of the L-histidine or its D-histidine enantiomer or its racemic mixture, or one of its isotopically marked derivatives or one of its a-N-methylated derivatives, or indirectly from the di-methylated derivative.

According to another specific embodiment of the method according to the invention, the controlled pH conditions are chosen in the interval pH=9-13 by adding an inorganic base such as sodium hydroxide, potassium or lithium hydroxide. It is important to note that a lower pH, for example pH=8.5, must be avoided subject to significantly lower selectivity of the reaction and a high yield of per-methylated hercynine ≧20%.

According to another specific embodiment of the method according to the invention, the mixture of polar solvents could be a methanol/water mixture.

According to another advantageous embodiment of the method according to the invention, the electrodialysis is done by using different membranes such as anion-selective membranes (for example, PC SA) and cation-selective membranes (for example, PC MV) in particular available from PC Cell (Germany).

The subsequent reactions making it possible to purify and recrystallize the L-hercynine to separate it from the organic impurities, from the electrodialysis filtrate, are done under traditional conditions, known by those skilled in the art.

According to another alternative embodiment, the method according to the invention is characterized in that it is possible to produce the L-hercynine-d9 from a reaction mixture resulting from the reaction of the L-histidine with a tri-deuterated (d3) methylation agent, such as iodomethane-d3.

According to a third aspect, the invention provides a new pure L-hercynine-d9 compound, with formula

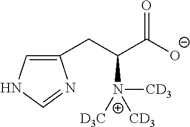

, in particular as obtained by the method defined in the preceding description or the following description including the examples.

It is known from the Foster document in “Advances in Drug Research, Academic Press, London, GB, Vol. 14, pp. 1-40, listed under no. XP009086953, ISSN 0065-2490” to deuterate pharmaceutical compounds. However, Foster only shows that deuterated medicaments make it possible to identify the metabolites during biodegradation of the modules.

In the context of the present invention, hercynine-D9 is specifically prepared, and for the first time, in which the D atoms are only on the part bonded to the nitrogen atom in the alpha position. The selective preparation in a tri-methylation step with iodomethane d3 followed by electrodialysis makes it possible to obtain the product easily, cost-effectively and with a better performance, compared to the alternative two-step preparation. This two-step preparation would be obvious for one skilled in the art by analogy with the preparation of non-deuterated hercynine according to the REINHOLD et al. protocol, in Journal of Medicinal Chemistry vol. 11, no. 2, Mar. 1, 1968, pages 258-260. However, this two-step preparation is not economically viable because it requires the use of three reagents containing deuterium: 1. reducing amination with formaldehyde-D2 is necessary, using gaseous deuterium D2 followed by quaternization with iodomethane-D3. Hercynine-D9 also has its own intrinsic properties.

The method according to the present invention, according to each of its aspects, has the advantage of not using dangerous reagents such as hydrogen, or using large quantities of ion exchange resins, as in the method of the prior art (Reinhold, V. et al.; J. Med. Chem.; 1968; 11; 258-260). It can also be done on a large scale, in non-toxic solvents such as water or a water/alcohol mixture. Aside from the feasibility aspect, these advantages considerably reduce not only the implementation costs, but also the negative impact on the environment.

The technical problems set out above are resolved for the first time all at once by the present invention, very easily and cost-effectively, the method for producing said new compounds being very easy to implement while providing good yields.

### DESCRIPTION OF THE FIGURES

FIG. 1: Chromatogram (HPLC) of the L-hercynine obtained in example 2 on an industrial scale after desalination and recrystallization (ELSD Universal detection)

FIG. 2: Chromatogram (HPLC) of the L-hercynine-d9 obtained in example 3 after desalination and recrystallization (ELSD Universal detection)

### EXAMPLES

The examples below, as well as the figures, are provided simply as an illustration and cannot in any way limit the scope of the invention, but are instead an integral part of the invention, in their general means.

In the examples described below, all percentages are given by weight, the temperature is room temperature or is given in degrees Celsius, and the pressure is the atmospheric pressure, unless otherwise stated. Room temperature refers to a temperature comprised between 20° C. and 35° C.

The reagents used are commercially available from international suppliers such as Sigma-Aldrich France (France), Alfa Aesar, Fisher Scientific, TCI Europe, Bachem (Switzerland), except the following compounds, which have been produced according to the cited protocol: N,N-Dimethyl-L-histidine hydrochloride hydrate (Reinhold, V. et al.; J. Med. Chem.; 1968; 11; 258-260).

The desalination was done by electrodialysis using cells made up of alternating anion-selective (for example, PC SA) and cation-selective (for example, PC MV) membranes in particular available from PC Cell (Germany), using desalination units like the ED 8002-001 unit (micro-unit for scale up to 10 g), the B-ED 1-3/ED 200 unit (up to 200 g) and the P20 unit (industrial scale), also available from PC Cell (Germany).

The NMR-¹H analyses were recorded at 400 MHz in D₂O or a D₂O/DCI mixture, using the HOD signal (4.79 ppm) as internal reference. The chemical displacements are noted in ppm, and the multiplicity of the signals indicated by the following symbols: s (singlet), d (doublet), t (triplet), q (quartet), and m (multiplet). The coupling constraints are noted in Hertz (Hz). The NMR-¹³C analyses are recorded at 75 MHz in the D₂O. The mass analyses are obtained by atmospheric pressure chemical ionization (APCI-MS). The melting points were measured with a device by the company Stuart Scientific. The HPLC analyses were done on an Acquity (Waters) device, using a C8 250x4.6 (5 μm) column. The mobile phase used is a water/acetonitrile mixture (98:2) in 12 min. and a flow rate of 0.6 mL/min. The detection is done with an ELSD (Sedere) universal detector.

### I. EXAMPLES ACCORDING TO THE INVENTION OF THE METHOD FOR PRODUCING PURE COMPOUNDS

The following examples illustrate a new, simple and reproducible method for producing pure L-hercynine, or its enantiomer, D-hercynine, or its racemic mixture, or one of its isotopically marked derivatives that is based on a method for separating inorganic salts, present in the reaction medium, by electrodialysis of an aqueous solution adjusted to pH=9, irrespective of the methylation mode used for the L-histidine or one of its methylated derivatives, followed by purification via recrystallization.

### Example 1 of the invention

### Isolation of the Pure L-Hercynine from a Reaction Mixture Obtained by Tri-Methylation of the L-Histidine

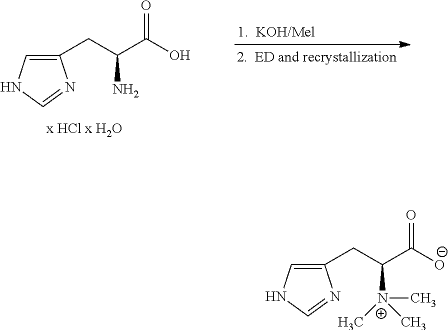

275 g (1.3 mol) of the hydrochloride hydrate of the L-histidine is solubilized in 2.7 L of a water/methanol mixture (2:1). The pH is adjusted to 9-13 with a 3M potassium hydroxide solution. Next, 645 g (4.55 mol) of iodomethane is added under agitation, and the mixture thus obtained is agitated for 18 hours at 25-35° C. while keeping the pH between 9-13. Next, the mixture is neutralized by adding 37% aqueous HC1.

After evaporation of the methanol, the pH of the aqueous solution, containing the raw product in mixture with the potassium chloride and iodide salts, is adjusted to 9 with a solution of NaOH (30%), then the solution is desalinated by electrodialysis until a conductivity of 50-100 μS is obtained. A test for the presence of halogen with silver nitrate, done on a withdrawn sample, confirms the absence of chloride and iodide ions.

The aqueous solution, obtained after electrodialysis, is dry evaporated. The raw product is purified by recrystallization in aqueous isopropanol, and the L-hercynine is obtained after filtration and drying (192 g; 73%) in the form of a white powder.
[α]D=+44.5° (c=1.0; 5N HC1)
pF: 238° C. (dec.)
NMR-¹H (D₂O, 400 MHz):β (ppm)=3.24 (m+s, 11H), 3.90 (dd, J=10 Hz, J=5 Hz, 1H), 6.98 (s, 1H), 7.69 (s, 1H).
NMR-¹H (D₂O/DCI, 400 MHZ):β (ppm)=3.31 (s, 9H), 3.44 (dd, J=14 Hz, J=12 Hz, 1H), 3.55 (dd, J=14 Hz, J=4 Hz, 1H), 4.13 (dd, J=12 Hz, J=4 Hz, 1H), 7.37 (s, 1H), 8.66 (s, 1H).
NMR-¹³C (D₂O, 75 MHz):β (ppm)=25.7; 52.4; 78.9; 116.7; 132.3; 136.5; 171.2. HPLC-MS (AP+): m/z=198 (MH+)
Purity by HPLC (ELSD detection): 100%

### Example 2 of the Invention

### Isolation of the Pure L-Hercynine from a Reaction Medium Obtained by Tri-Methylation of the L-Histidine on the Industrial Scale

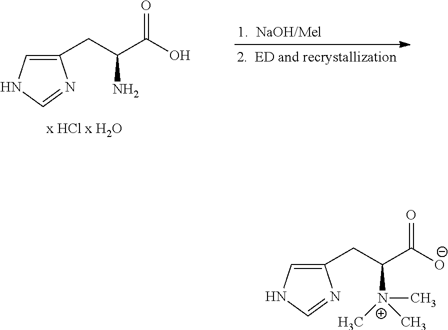

48.0 kg (229 mol) of the hydrochloride hydrate of the L-histidine is solubilized in 475 liters of a water/methanol mixture (2:1). The pH is adjusted to 9-13 with a 10% sodium hydroxide solution. Next, 113.8 kg (801 mol) of iodimethane is added under agitation, and the mixture thus obtained is agitated for 18 hours at 25-35° C. while keeping the pH between 9-13. Next, the mixture is neutralized by adding 37% aqueous HC1.

After evaporation of the methanol, the pH of the aqueous solution, containing the raw product in mixture with the sodium chloride and iodide salts, is adjusted to 9 with a solution of NaOH (30%), then this solution is desalinated by electrodialysis until a conductivity of 50-100 μS is obtained. A test for the presence of halogen with silver nitrate, done on a withdrawn sample, confirms the absence of chloride and iodide ions.

The desalinated aqueous solution, obtained after electrodialysis, is concentrated. After recrystallization in aqueous isopropyl, the L-hercynine is obtained after filtration and drying (32.2 kg; 72%) in the form of a white powder.
[α]D:+44° (c=1.0; 5N HC1)
pF: 242° C. (dec.)

The NMR-¹H and NMR-¹³C and mass spectrums are identical to those of the L-hercynine obtained in example 1.

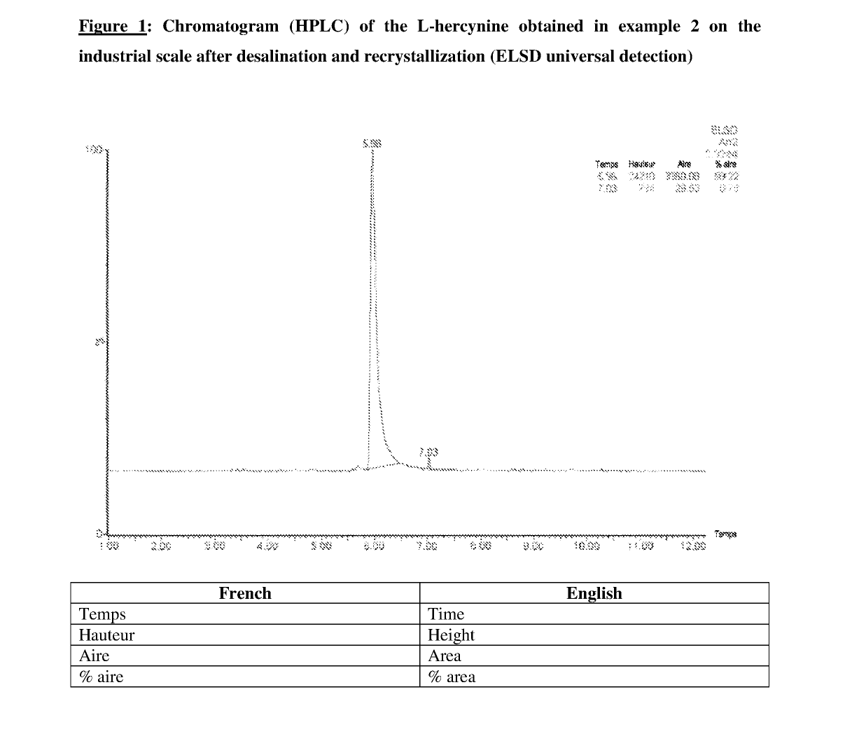

Purity by HPLC (ELSD detection): 99.2% (see FIG. 1).

### Example 3 of the Invention

### Isolation of the Pure L-Hercynine-d9 From the Reaction Mixture Obtained by Tri-Methylation of the L-Histidine

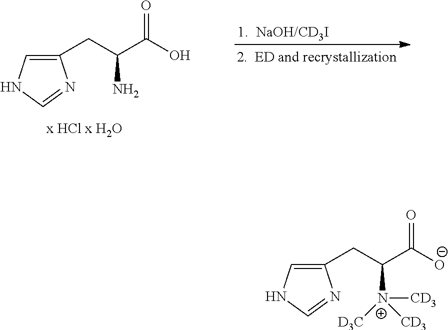 [SMILES: N[C@@H](Cc1c[nH]cn1)C(=O)O ; [2H]C([2H])([2H])[N+]([C@@H](Cc1c[nH]cn1)C(=O)[O-])(C([2H])([2H])[2H])C([2H])([2H])[2H]]

31.4 g (150 mmol) of the hydrochloride hydrate of the L-histidine is solubilized in 300 mL of a water/methanol mixture (2:1). The pH is adjusted to 9-13 with a 3M sodium hydroxide solution. Next, 86.9 g (600 mmol) of iodomethane-d3 is gradually added under agitation, while keeping the pH between 9-13. After 18 hours of agitation at 30° C., the mixture thus obtained is neutralized by adding 37% aqueous hydrochloric acid.

After evaporation of the methanol, the pH of the aqueous solution, containing the raw product in mixture with the sodium chloride and iodide salts, is adjusted to 9 with a NaOH solution (30%), then the solution is desalinated by electrodialysis until obtaining a conductivity of 50-100 μS. A test for the presence of halogen with silver nitrate, done on a withdrawn sample, confirms the absence of chloride and iodide ions.

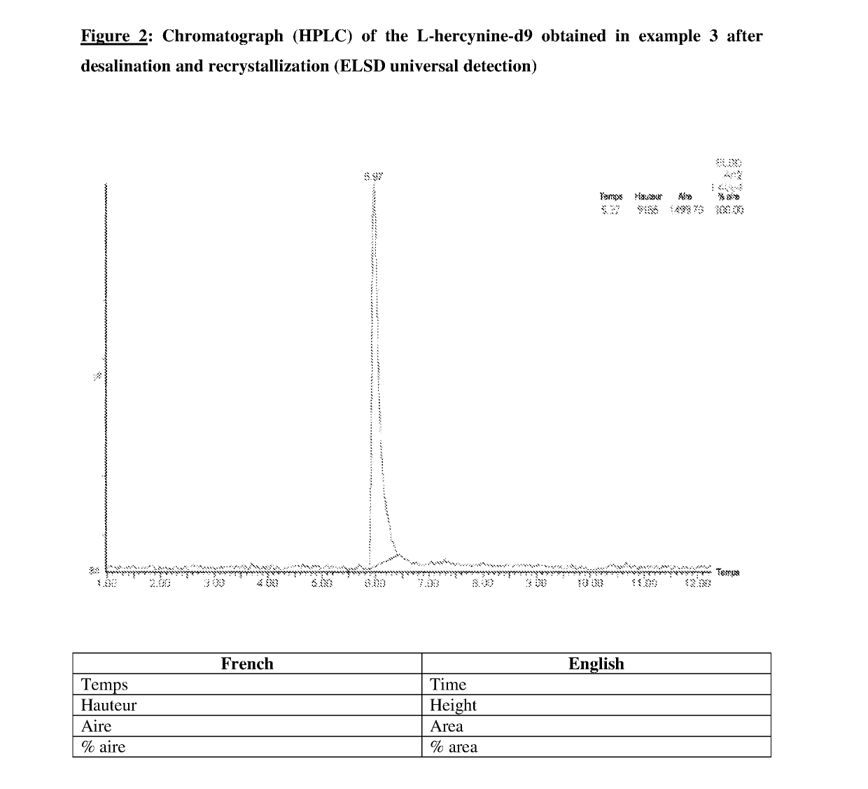

The aqueous solution, obtained after electrodialysis, is dry evaporated. The obtained raw product is purified by recrystallization in the aqueous isopropanol, and the L-Hercynine-d9 is obtained after filtration and drying (23.8 g; 77%) in the form of a white powder.
NMR-¹H (D₂O/DCI, 400 MHz):β (ppm)=3.14 (m, 2H); 3.83 (dd, J=10 Hz, J=5 Hz, 1H); 6.92 (s, 1H), 7.62 (s, 1H).
HPLC-MS (AP+): m/z=207 (MH+).
Purity by HPLC (ELSD detection): 100% (see FIG. 2).

### Example 4 of the Invention

### Isolation of the Pure L-Hercynine from a Reaction Mixture Obtained by Quaternization of the N,N-Dimethyl-Histidine with Iodomethane

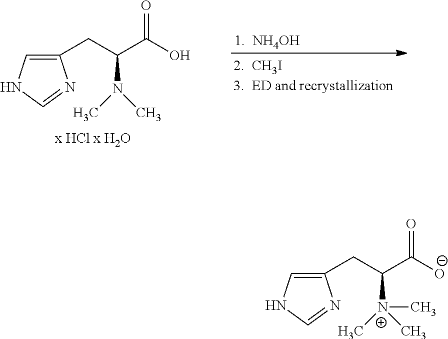

The N,N-dimethyl-histidine is quaternized as described by Reinhold, V. et al.; J. Med. Chem.; 1968; 11; 258-260. In short, 48.5 g (200 mmol) of the hydrochloride hydrate of the N,N-dimethyl-histidine is solubilized in 1 liter of methanol. The pH is adjusted to 9 with a 20% ammonia aqueous solution. Next, 37.3 g (16 mL, 1.3 equivalents) of iodomethane is added under agitation. The solution thus obtained is agitated for 18 hours at 30° C. (room temperature). After evaporation of the methanol, the raw product, in the reaction mixture containing the ammonium chloride and iodide salts (88 g), is solubilized in 500 mL of water, the pH is adjusted to 9 with a NaOH (30%) solution, then the solution is desalinated by electrodialysis until a conductivity of 43 microsiemens (μS) is obtained. A test with silver nitrate, making it possible to estimate the presence of halogenide ions, done on a sample taken from the aqueous solution, confirms the absence of chloride and iodide ions.

The aqueous solution, obtained after electrodialysis, is dry evaporated. The raw product obtained is purified by recrystallization, and the L-hercynine is obtained after filtration and drying (36.6 g; 93%) in the form of a white powder.
[α]D=+44° (c=1.0; 5N HC1)
pF: 241° C. (dec.)
NMR-¹H (D₂O, 400 MHz):β (ppm)=3.16 (m+s, 11H), 3.85 (dd, J=10 Hz, J=5 Hz, 1H), 6.93 (s, 1H), 7.64 (s, 1H).
NMR-¹³C (D₂O, 75 MHz):β (ppm)=25.7; 52.4; 78.9; 116.7; 132.3; 136.5; 171.2.
HPLC-MS (AP+): m/z=198 (MH+)

### Example 5 of the Invention

### Isolation of the Pure D-Hercynine from a Reaction Mixture Obtained by Quaternization of the D-N,N-Dimethyl-Histidine with Iodomethane

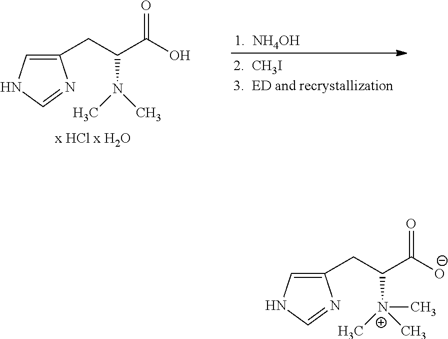

The D-N,N-dimethyl-histidine is quaternized by analogy with example 4 describing the synthesis and purification of the L-hercynine. In short, 21.3 g (90 mmol) of the hydrochloric hydrate of the D-N,N-dimethyl-histidine is solubilized in 0.45 liters of methanol. The pH is adjusted to 9 with a 20% ammonia aqueous solution. Next, 15.3 g (6.7 mL, 1.2 equivalents) of iodomethane is added under agitation. The solution thus obtained is agitated for 18 hours at 25-30° C. After evaporation of the methanol, the raw product, in the reaction mixture containing the ammonium chloride and iodide salts, is solubilized in 300 mL of water. After adjustment of the pH of the solution to 9 with a NaOH (30%) solution, the mixture is desalinated by electrodialysis until a conductivity of 70 μS is obtained. A silver nitrate test, making it possible to estimate the presence of halogenide ions, done on a sample taken from the aqueous solution, confirms the absence of chloride and iodide ions.

The aqueous solution, obtained after electrodialysis, is dry evaporated. The raw product obtained is purified by recrystallization with a methanol/ethyl acetate mixture, and the L-hercynine is obtained after filtration and drying (16.7 g; 93%) in the form of a white powder.
[α]D: -44° (c=1.0; 5N HC1)
The NMR-¹H and NMR-¹³C and mass spectrums are identical to those of the L-hercynine obtained in example 1.

### Example 6 of the Invention

### Isolation of the Pure L-Hercynine from a Reaction Mixture Obtained by Quaternization of the N,N-Dimethyl-Histidine with the Methyl Sulfate

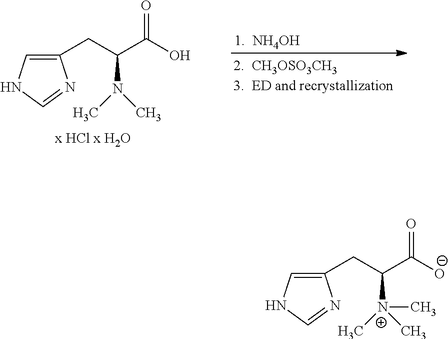

The N,N-dimethyl-histidine is quaternized by analogy with the procedure described by Reinhold, V. et al.; J. Med. Chem.; 1968; 11; 258-260, replacing the iodomethane with methyl sulfate. In short, 4.85 g (20 mmol) of hydrochloride hydrate of the N,N-dimethyl-histidine is solubilized in 100 mL of methanol. The pH is adjusted to 9 with a 20% ammonia aqueous solution. Next, 3.03 g (2.28 mL, 24 mmol, 1.2 equivalents) of methyl sulfate is added under agitation. The solution thus obtained is agitated for 18 hours at 30° C. The NMR-¹H analysis of a sample indicates that the conversion is not complete. 1.0 g (0.75 mL, 8 mmol, 0.4 equivalents) of methyl sulfate is added to the reaction mixture. After agitation for 3 hours, the solvent is evaporated, and the raw product, in the reaction mixture containing the ammonium chloride and iodide salts, is solubilized in 50 mL of water. After adjusting the pH to 9, desalination by electrodialysis is done until a conductivity of 50 μS is obtained, a silver nitrate test making it possible to estimate the presence of halogenide ions, done on a sample taken from the aqueous solution, and an analysis by HPLC-ELSD confirms the absence of salts.

The aqueous solution, obtained after electrodialysis, is dry evaporated. After recrystallization of the raw product with a methanol/ethyl acetate mixture, the L-hercynine is obtained after filtration and drying (3.0 g; 76%) in the form of a white powder.

The NMR-¹H spectrum and the rotatory power are identical to those obtained for the isolated product in example 1.

### II. COMPARATIVE EXAMPLES OF THE ELECTRODIALYSIS METHOD

### Comparative Example 1

### Evaluation of the Separation of the Inorganic Salts from the L-Hercynine at pH 7 and pH 8

A) 2000 mL of an aqueous solution containing 113 g (0.57 mol) L-hercynine, 300 g (3.5 equiv.) of sodium iodide and 33 g (1 equiv.) of sodium chloride (1.3 mol) at pH 7 and a conductivity of 70 mS/cm are placed in the “diluent” compartment of an electrodialysis device, for example the B-ED 1-3/ED 200 unit, equipped with an ED 200 cell. 2000 mL of demineralized water is placed in the “concentrate” compartment. 8000 mL of an aqueous solution of sodium sulfate (0.5-1%) is used as electrolyte. The circulation flow rate of all of the solutions is 100/120 L/h, by applying a voltage of 10-15 V, until the conductivity of the “diluent” is <0.1 mS/cm. A test for the presence of halogen with the silver nitrate, done on a withdrawn sample, confirms the absence of chloride and iodide ions. The “diluent” contains 86 g of L-hercynine, corresponding to a loss of 24%.

B) 2000 mL of an aqueous solution containing 113 g (0.57 mol) of L-hercynine, 300 g (3.5 equiv.) of sodium iodide and 33 g (1 equiv.) of sodium chloride (1.3 mol) at pH 8 and a conductivity of 75 mS/cm is electrodialyzed (as described in A) up to a conductivity of the “diluent” of 0.02 mS/cm. A test for the presence of halogen with silver nitrate, done on a withdrawn sample, confirms the absence of chloride and iodide ions. The diluent contains 98.3 g of L-hercynine, corresponding to a loss of 13%.

### Comparative Example 2

### Evaluation of the Separation of the Inorganic Salts from the L-Hercynine at pH 7 (Comparative) and pH 9 (According to the Invention)

A) The reaction mixture obtained by tri-methylation of the L-histidine, as described in example 2 of the invention, after neutralization with the hydrochloric acid HC1, containing 5% (w/w) of the hercynine mixed with reaction sub-products, as well as the sodium chloride and iodide salts, is desalinated in portion in 2000 mL by electrodialysis as described in comparative example 1A, until a conductivity of 50-100 μS is obtained. The L-hercynine content is assayed by HPLC. The procedure has been applied on 5 lots numbered 1 to 5, and the L-hercynine losses (from 22-25%) are listed in table 1.

B) Under the same conditions, with the exception that the pH is adjusted to 9, according to the invention, with 30% sodium hydroxide in the “diluent” compartment upon starting the electrodialysis, 5 lots numbered 6 to 10 are desalinated until obtaining a conductivity of 50-100 μS. The L-hercynine content in the “diluent” is assayed by HPLC. The procedure was applied on 5 lots, and the L-hercynine losses, going from only 1.5-2.5%, are listed in Table 1.

TABLE 1
L-hercynine losse

| | | Lot 1 | Lot 2 | Lot 3 | Lot 4 | Lot 5 |
|---|---|---|---|---|---|---|
| Example | loss | 24% | 22% | 25% | 22% | 23% |
| 2A: pH = 7 | | | | | | |
| | | Lot 6 | Lot 7 | Lot 8 | Lot 9 | Lot 10 |
| Example | loss | 2.5% | 2.3% | 1.9% | 2% | 1.5% |
| 2B: pH = 9 | | | | | | |

Comparative examples 1 and 2 therefore indeed show that the losses during electrodialysis can be limited very significantly by adjusting the pH of the aqueous solution to 8.5-9.5, ideally to around 9, at the beginning of the electrodialysis, according to the invention.

1. A method for purifying L-hercynine, or its enantiomer, D-hercynine, or its racemic mixture, or one of its isotopically marked derivatives from a reaction mixture resulting from the reaction of L-histidine, or its enantiomer D-histidine or its racemic mixture, or one of its isotopically marked derivatives or one of its a-N-methylated derivatives, under controlled pH conditions, with a methylation agent MeX in a polar solvent or a mixture of polar solvents, at room temperature, characterized in that it comprises at least one step for separating the organic products from the inorganic salts formed during the reaction by electrodialysis; and in that the electrodialysis is conducted on a reaction medium adjusted to a pH comprised between 8.5 and 9.5, at the beginning of the electrodialysis.

2. The purification method according to claim 1, characterized in that it comprises an additional step for recrystallization of the L-hercynine, or its enantiomer, D-hercynine, or its racemic mixture, or one of its isotopically marked derivatives or one of its methylated derivatives, to eliminate the organic impurities.

3. A method for the industrial-scale production of L-hercynine, or its enantiomer, D-hercynine, or its racemic mixture, or one of its isotopically marked derivatives, or one of its a-N-methylated derivatives, characterized in that it comprises the following steps:
reacting the L-histidine, or its enantiomer D-histidine or its racemic mixture, or one of its isotopically marked derivatives or one of its a-N-methylated derivatives, under controlled pH conditions, with a methylation agent MeX in a polar solvent or a mixture of polar solvents, at room temperature; followed by
separating the organic products from the inorganic salts formed during the reaction by electrodialysis; and in that the electrodialysis is done on a reaction medium adjusted to a pH comprised between 8.5 and 9.5, upon starting the electrodialysis; followed by
re-crystallizing the L-hercynine to eliminate the organic impurities.

4. The method according to claim 1, characterized in that the electrodialysis is done on a reaction medium adjusted to about 9, at the beginning of the electrodialysis.

5. The method according to claim 1, characterized in that the methylating agent MeX is chosen from among methyl halogenide, methyl sulfate, methyl carbonate or methyl or trifluoromethyl or tosyl sulfonate.

6. The method according to claim 1, characterized in that the polar solvent or the mixture of polar solvents is chosen from among water; a C1—C6 alcohol, for example methanol, ethanol, propanol or propanol-2, butanol; ethyl acetate. One particular polar mixture is a water/alcohol, water/propanol or propanol-2, methanol/ethyl acetate mixture.

7. The method according to claim 1 one of the claims 1 to 6, characterized in that the controlled pH conditions are chosen to result directly in a tri-methylation of the L-histidine or its D-histidine enantiomer or its racemic mixture, or one of its isotopically marked derivatives or one of its a-N-methylated derivatives, or indirectly from the di-methylated derivative.

8. The method according to claim 1, characterized in that the controlled pH conditions are chosen in the interval pH=9-13 by adding an inorganic base such as sodium hydroxide, potassium or lithium hydroxide.

9. The method according to claim 1, characterized in that the L-Hercynine-d9 is prepared from a reaction mixture resulting from the reaction of the L-Histidine with a methylation-d3 agent, in particular iodomethane-d3.

10. L-Hercynine-d9, with formula 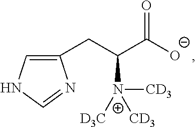
in particular as obtained using the method according to claim 1.
